(19) United States
(12) Patent Application Publication
(10) Pub. No.: US 2016/0081557 A1
(43) Pub. Date: Mar. 24, 2016

(54) PHOTOACOUSTIC IMAGING PROBE

(71) Applicant: ALPINION MEDICAL SYSTEMS CO., LTD., Hwaseong-si, Gyeonggi-do (KR)

(72) Inventors: Susung LEE, Yongin-si (KR); Heewon KIM, Seoul (KR)

(21) Appl. No.: 14/787,890

(22) PCT Filed: Apr. 30, 2013
(86) PCT No.: PCT/KR2013/003764

§ 371 Date: Oct. 29, 2015

(30) Foreign Application Priority Data
Apr. 30, 2013 (KR) 10-2013-0048797

(51) Int. Cl. A61B 5/00

(52) U.S. Cl. CPC A61B 5/0095

(57) ABSTRACT

Some embodiments of the present disclosure provide a photoacoustic imaging probe including a light transmitting unit configured to transmit light to a target subject to be inspected and an acoustic-wave receiving unit configured to receive an acoustic wave generated from the target subject by being irradiated with the light and to accommodate the light transmitting unit therein in a manner that a first side of the light transmitting unit is exposed to outside.

### TECHNICAL FIELD

Some embodiments of the present disclosure relate to a photoacoustic imaging probe. More particularly, some embodiments of the present disclosure relate to a probe for acquiring an image of a target subject to be inspected, by using a photoacoustic effect.

### BACKGROUND

The statements in this section merely provide background information related to some embodiments of the present disclosure and do not necessarily constitute prior art.

Photoacoustic effect refers to an excitation effect of acoustic waves from a subject when irradiated with light.

Photoacoustic imaging techniques that utilize such photoacoustic effect involve illuminating a subject (living body) with the light emitted from a light source, receiving acoustic waves generated by the light as absorbed by living tissues in its propagation or diffusion through the subject, and visualizing the anatomical information inside the subject, a living body by analysis of the received acoustic waves.

Techniques utilizing the photoacoustic effect have been researched and developed in recent times. Such research and development have been actively conducted especially in the field of medical appliances.

FIG. 1 shows how light is transmitted by a probe using a photoacoustic imaging technology.

Referring to FIG. 1, the photoacoustic imaging technology is particularly applicable to, among others, methods of using a probe typically having a nose section with two side portions 1 and 2 for irradiating a subject with the convergence of light rays 3.

However, the methods cause a dead zone 4 to occur at a short distance devoid of projection of light, and they cannot be applied to where a shallow focal depth is involved, such as the diagnosis of superficial organs.

In addition, the conventional photoacoustic imaging probes have employed optical fibers for the configuration of irradiating light, which take up a substantial volume of the imaging probes and undesirably increase the production cost. Further, the optical fibers that reside internally of a bundle of cables would raise reliability issue over continuous bending of the cable due to repeated use.

### DISCLOSURE

### Technical Problem

Therefore, the present disclosure seeks to provide a photoacoustic imaging probe for use in medical and other applications, which has a new structure for precluding the creation of a dead zone of projection of light.

In addition, the present disclosure provides a photoacoustic imaging probe having a structure using an element operating at an electric signal, for a light source.

The aforementioned technical issues are not restrictive, and other unspecified technical problems to be addressed by the present disclosure should be clearly understood by a person having ordinary skill in the pertinent art, from the following description.

### SUMMARY

A photoacoustic imaging probe according to some embodiments of the present disclosure includes a light transmitting unit configured to transmit light to a target subject to be inspected and an acoustic-wave receiving unit configured to receive an acoustic wave generated from the target subject by being irradiated with the light and to accommodate the light transmitting unit therein in a manner that a first side of the light transmitting unit is exposed to outside.

A medical apparatus employing an acoustic wave, according to some embodiments of the present disclosure includes the above-mentioned photoacoustic imaging probe.

A method of manufacturing a photoacoustic imaging probe, according to some embodiments of the present disclosure, includes laminating a piezoelectric member and an acoustic-impedance matching layer on a backing layer or sound absorbing layer, the sound absorbing layer, the piezoelectric member, and the acoustic-impedance matching layer laminated in this order defining a laminated member, multi-dicing the laminated member in a second direction perpendicular to a first direction along which a plurality of oscillators is arranged at intervals, forming a groove elongated along the first direction on a diced laminated member, inserting a light guide unit into the groove, and forming a lens layer so that a first side of the light guide unit is exposed to outside.

A medical apparatus employing an acoustic wave, according to some embodiments of the present disclosure, includes a photoacoustic imaging probe manufactured by the above-mentioned method.

### Advantageous Effects

According to the present disclosure, a photoacoustic imaging probe includes a light transmitting unit which is inserted in a transducer, eliminating in effect the creation of a dead zone of light projection from the front of the probe over to an imaging plain.

In addition, an element operating at an electric signal is employed as a light source, which reduces the manufacturing cost of the relevant apparatuses while allowing the use of the existing cable for ultrasonic probes free from the reliability issue that would be raised if optical fibers were used.

Besides, different embodiments of the present disclosure have a variety of effects such as having excellent durability, which can be identified by the following description of the embodiments.

### BRIEF DESCRIPTION OF DRAWINGS

FIG. 1 is a diagram of light transmitted by a photoacoustic imaging probe according to the prior art.

FIG. 2 is a conceptual perspective view of a photoacoustic imaging probe according to some embodiments of the present disclosure.

FIG. 3 is a conceptual side view of a photoacoustic imaging probe according to some embodiments of the present disclosure.

FIG. 4 is a conceptual view of the photoacoustic imaging probe of FIG. 3 showing a light guide unit tapered in a predetermined direction toward a first side.

FIGS. 5A to 5C are plan views of an acoustic-wave receiving unit formed with one or more through-holes for accommodating at least one light unit.

FIG. 6 is a perspective view of a housing which encloses a light transmitting unit and an acoustic-wave receiving unit according to some embodiments of the present disclosure.

FIG. 7 is a flowchart of a method of manufacturing a photoacoustic imaging probe according to some embodiments of the present disclosure.

### REFERENCE NUMERALS

| | |
|---|---|
| 10: photoacoustic imaging probe | 20: light transmitting unit |
| 30: acoustic-wave receiving unit | C: centerline |

### DETAILED DESCRIPTION

Exemplary embodiments of the present disclosure are described in detail below with reference to the accompanying drawings. In the following descriptions, like reference numerals designate like elements although the elements are shown in different drawings. Further, detailed descriptions of known functions and configurations incorporated herein are omitted for the purpose of clarity and for brevity.

Additionally, various terms such as first, second, A, B, etc., are used solely for the purpose of differentiating one component from the other but not to imply or suggest the substances, the order or sequence of the components. If a component were described as ‘connected’, ‘coupled’, or ‘linked’ to another component, they may mean the components are not only directly ‘connected’, ‘coupled’, or ‘linked’ but also are indirectly ‘connected’, ‘coupled’, or ‘linked’ via one or more additional components.

In the present disclosure, the acoustic wave includes sound wave, ultrasonic wave, and photoacoustic wave among others and may refer to elastic wave or the like which is generated internally of the subject by transmitting light (electromagnetic wave) such as near infrared to the subject.

In addition, the photoacoustic imaging probe according to some embodiments of the present disclosure can be used for diagnosis of malignant tumors and vascular diseases of, for example, humans and animals, and it may be an apparatus for acquiring biological information from the inside of the subject and generating image data. Thus, the subject may refer to the target site of the diagnosis such as a breast of the human body or animals, a finger or limbs.

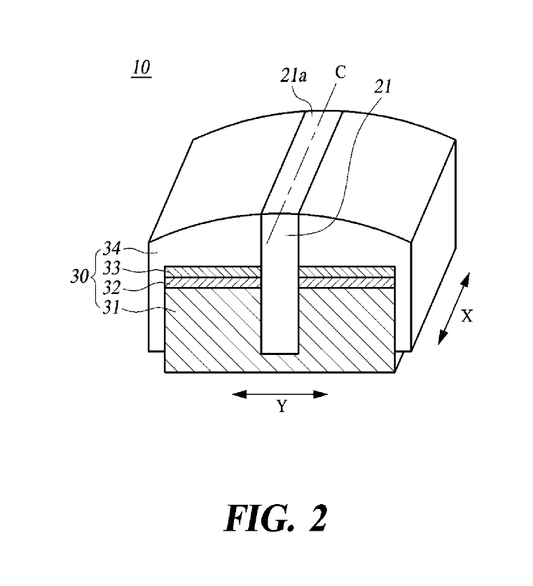

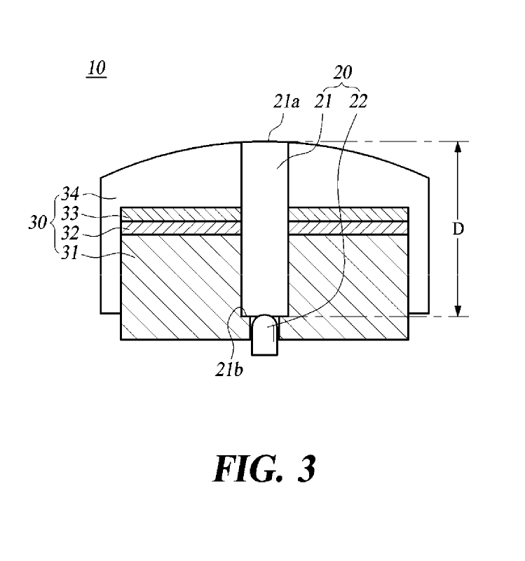

FIG. 2 is a conceptual perspective view of a photoacoustic imaging probe 10 according to some embodiments of the present disclosure. FIG. 3 is a conceptual side view of the photoacoustic imaging probe 10 according to some embodiments of the present disclosure. An embodiment will be described with reference to FIGS. 2 and 3.

The photoacoustic imaging probe 10 according to embodiments is configured to include a light transmitting unit 20 and an acoustic-wave receiving unit 30.

The light transmitting unit 20 may serve to transmit light to the subject. Light transmitting unit 20 may be configured to have a light guide unit 21 and at least one light unit 22.

The light guide unit 21 may serve to guide the light irradiated from the light unit 22 towards the subject under test. The light guide unit 21 may include an optically transparent material. The optically transparent material is not necessarily meant to be transparent to the naked eye, but intended to include all materials capable of guiding light. The light guide unit 21 can be, for example, a glass material or a transparent plastic material.

The light unit 22 may serve to irradiate the light to the light guide unit 21.

The light irradiated by the light unit 22 may have a predetermined wavelength that is absorbed by the subject, namely hemoglobin or other predetermined components constituting a living body. Depending on the embodiments, the wavelength of light may be 500 nm to 1500 nm. The wavelengths in this range make it easy to distinguish the acoustic waves generated from the light-absorbing material inside of the subject from those from the skin. Here, the light-absorbing material inside of the subject may be blood vessel carrying oxidized or reduced hemoglobin or a malignant tumor with neovascularization.

In some embodiments, the light unit 22 may be operated by an electrical signal. In embodiments, the light unit 22 may be a light-emitting diode as well as a laser diode capable of irradiating a laser beam.

Operating the light unit 22 with electric signals can be simply implemented, which reduces the manufacturing cost of the photoacoustic imaging probe 10, and the method takes less volume within the probe to provide a designing advantage of the probe structure with efficiency.

The acoustic-wave receiving unit 30 is configured to receive acoustic waves which are generated by the subject in response to the transmitted light. In some embodiments, the acoustic-wave receiving unit 30 may incorporate a transducer 30 capable of receiving the acoustic waves and converting them into electric signals. At least one embodiment of the transducer 30 may be constructed by laminating a sound absorbing layer 31, a piezoelectric member 32 and an acoustic-impedance matching layer 33 into a laminated member with a lens layer 34 formed on the front surface of the acoustic-impedance matching layer 33.

Here, the piezoelectric member 32 refers to a substance that generates a piezoelectric effect. The sound absorbing layer 31 is to suppress the free vibration of the piezoelectric member 32, thereby reducing the pulse width of the acoustic wave, to stop the acoustic waves from unnecessarily traveling behind the piezoelectric member 32 and in turn prevents an image distortion. The acoustic-impedance matching layer 33 serves to match the acoustic impedance of the piezoelectric member 32 with that of the subject.

At least one embodiment of the transducer 30 may be made by laminating the piezoelectric member 32 and the acoustic-impedance matching layer 33 on the sound absorbing layer 31 into a laminated member, followed by multi-dicing the laminated member in a second direction (Y-direction) perpendicular to a first direction (X-direction) along which a plurality of oscillators is arranged at intervals, and forming a lens layer frontally of the acoustic-impedance matching layer 33. Here, the plurality of oscillators may be a number of separate elements which are electrically or otherwise independent from each other to be able to individually receive the acoustic waves.

One of features of the present disclosure is that the light transmitting unit 20 is disposed within the acoustic-wave receiving unit.

In embodiments, the photoacoustic imaging probe 10 may be configured so that the acoustic-wave receiving unit 30 is formed centrally with a groove, elongated along the first direction (X-direction), in which the light transmitting unit 20 is disposed. In other words, the photoacoustic imaging probe 10 features that the light transmitting unit 20 is longitudinally colinear with the first direction (X-direction) of the acoustic-wave receiving unit or transducer 30 upon placing the light guide unit 21 therein.

It is noted that the light transmission unit 20 has a first side 21a exposed to outside of the photoacoustic imaging probe 10. Here, the first surface 21a refers to the outermost surface where light for transmission leaves the light transmitting unit 20.

In other words, the photoacoustic imaging probe 10 may be configured to have the first side 21a disposed at a centerline of the front side of the lens layer 34 of the acoustic-wave receiving unit or transducer 30. The first side 21a is formed in an elongated manner along the first direction (X-direction) of the lens layer 34. The light guide unit 21 including the first side 21a may be referred to as an optical window 21. Light can be transmitted through the light guide unit 21. The remaining portion except the first surface of 21a, that is, the lens layer 34, may be referred to as an acoustic window 34. Acoustic waves may be received through the lens layer 34.

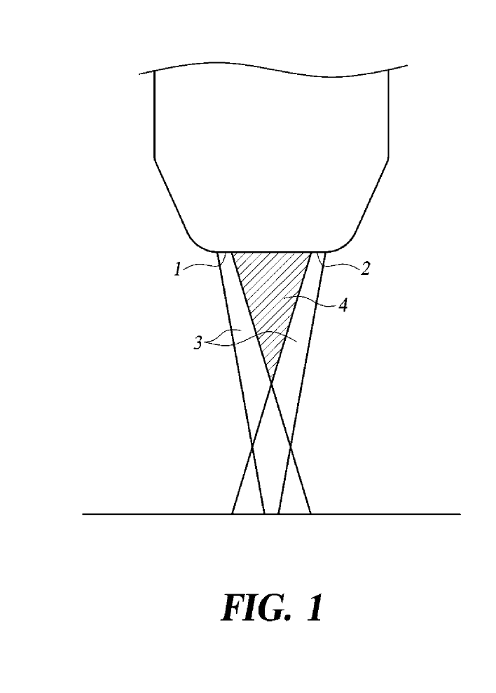

Referring to FIG. 1, a conventional photoacoustic imaging probe has transmitted light 3 in a form which are converged at a place from both sides 1 and 2. This has left a near field the dead zone 4 where no image could be obtained. However, the photoacoustic imaging probe 10 according to an embodiment has no such zone 4, and hence images can be obtained even at close range.

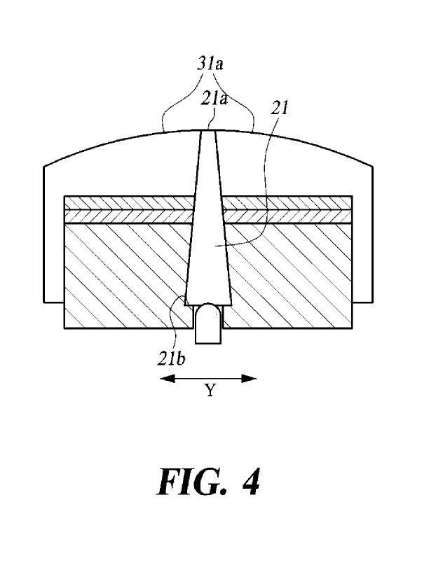

FIG. 4 illustrates the photoacoustic imaging probe 10, wherein the light guide unit 21 is tapered from the opposing side toward the first side 21a where the width of the light guide unit 21 along the second direction (Y-direction) is the narrowest.

An embodiment will be described with reference to FIGS. 3 and 4.

In some embodiments, the light guide unit 21 may have a constant cross sectional area from the opposing side 21b toward the first side 21a as shown in FIG. 3. According to another embodiment, the cross-sectional area thereof may not be constant. Depending on the embodiment, the cross-sectional area may be made narrower from the opposing side 21b to the first surface 21a. This is illustrated in FIG. 4 where the light guide unit 21 is tapered from the opposing side 21b to the first side 21a exhibiting the narrowest width along the second direction (Y-direction).

Tapered from the opposing side toward the first side 21a, the light guide unit 21 takes advantage of Huygen's principle which in effect spreads light in the second direction (Y-direction) as it passes through the first side 21a. This further provides the acoustic-wave receiving unit 30 with a relatively wider receive face 31a, resulting in improved reception sensitivity.

In embodiments, the depth D of the aforementioned groove may be up to a portion of the sound absorbing layer 31. The light unit 22 may be disposed in proximity of the opposing side 21b of the first side 21a of the light guide unit 21. In embodiments, there may be at least one through-hole formed on a bottom of the groove for inserting at least one light unit 22.

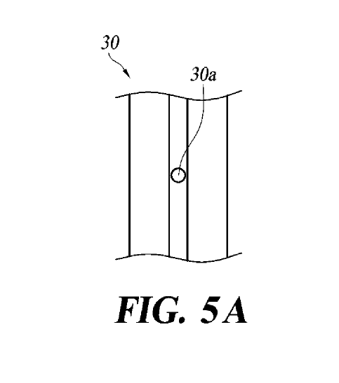

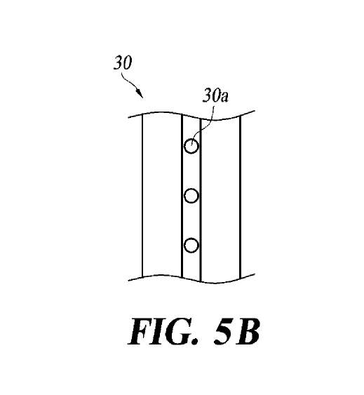

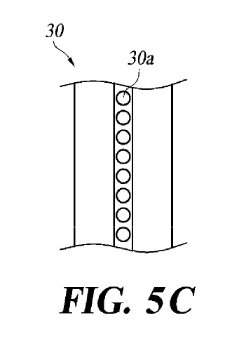

FIGS. 5A to 5C are plan views of the acoustic-wave receiving unit 30 formed with one or more through-holes 30a for accommodating at least one light unit 22. Embodiments will be described with reference to FIGS. 5A to 5C.

In embodiments, the bottom of the groove may be located in the sound absorption layer 31. In this case, the through-hole 30a for receiving the light unit 22 is formed within the sound absorption layer 31. As described above, the light unit 22 may be at least one. The light unit 22 according to embodiments may installed alone in the center of the groove, or the light units 22 may be installed successively or intermittently end to end along the longitudinal direction of the groove, although they may be otherwise installed in various patterns. FIG. 5A shows a single through-hole 30a installed, FIG. 5B three of those installed, and FIG. 5C eight through-holes 30a. The through-holes 30a may be arranged in a line along the groove as shown in FIGS. 5A-5C, or they may be arranged in different patterns. There may be full through-holes 30a formed along the longitudinal length of the groove end to end, as shown in FIG. 5C.

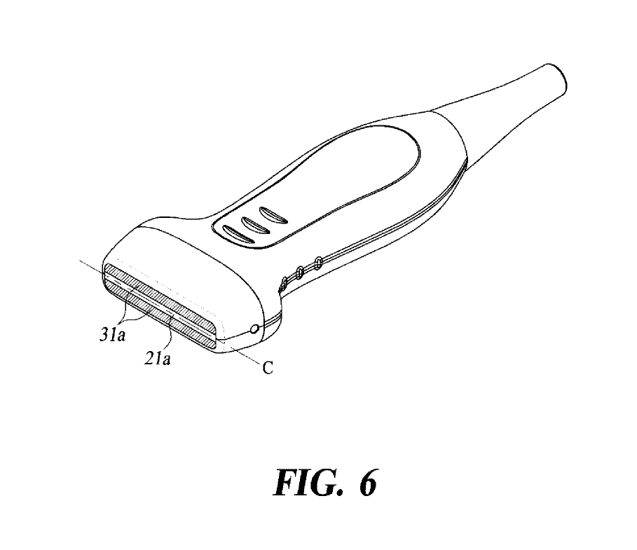

FIG. 6 is a perspective view of a housing which encloses the light transmitting unit 20 and the acoustic-wave receiving unit 30 according to some embodiments of the present disclosure. Referring to FIG. 6, the first surface 21a is shown as residing in the centerline C, and the receive face 31a is neighboring the acoustic-wave receiving unit 30.

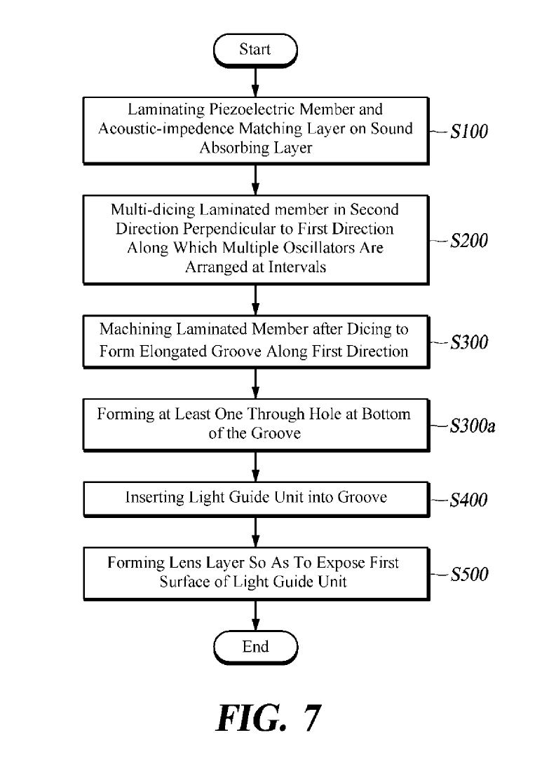

FIG. 7 is a flowchart of a method of manufacturing a photoacoustic imaging probe according to some embodiments of the present disclosure.

Referring to FIG. 7, the method for making a photoacoustic imaging probe may include laminating the piezoelectric member 32 and the acoustic-impedance matching layer 33 on the sound absorbing layer 31, the sound absorbing layer, the piezoelectric member, and the acoustic-impedance matching layer laminated in this order defining a laminated member (step S100). The method may further include multi-dicing the laminated member in the second direction (Y-direction) perpendicular to the first direction (X-direction) along which a plurality of oscillators is arranged at intervals (step S200). The method may further include forming a groove elongated along the first direction on the diced laminated member (step S300). The method may further include inserting the light guide unit 21 into the groove (step S400). The method may further include forming the lens layer 34 so that the first side 21a of the light guide unit 21 is exposed to outside (step S500).

The method for making a photoacoustic imaging probe according to some embodiments may feature that the inserting step (S400) includes inserting the light guide unit 21 so as to protrude from a surface of the acoustic-impedance matching layer 33. This feature facilitates the latter step of forming the lens layer 34 by using the protruded area as a guide to work around. Thereby, a structure is provided with the first side 21a disposed at the centerline C of the surface of the lens layer 34.

The method for making the photoacoustic imaging probe may further include forming at least one through-hole 30a on a bottom of the groove after the forming of the groove (step S300).

Meanwhile, some embodiments provide a medical apparatus employing an acoustic wave, which includes the photoacoustic imaging probe 10 according to the foregoing description. Medical apparatuses or devices of any kind can embody the present disclosure as long as they are available for application of the aforementioned photoacoustic imaging probe 10. Specific names of such medical apparatuses or devices are well known and they will not be elaborated herein.

The present disclosure should not be limited to these embodiments but various modifications and variations are made by one ordinarily skilled in the art within the subject matter, the idea and scope of the present disclosure as hereinafter claimed.

Specific terms used in this disclosure and drawings are used for illustrative purposes and not to be considered as limitations of the present disclosure. Exemplary embodiments of the present disclosure have been described for the sake of brevity and clarity. Accordingly, one of ordinary skill would understand the scope of the claimed invention is not to be limited by the explicitly described above embodiments but by the claims and equivalents thereof.

### CROSS-REFERENCE TO RELATED APPLICATION

If applicable, this application claims priority under 35 U.S.C §119(a) of Patent Application No. 10-2013-0048797, filed on Apr. 30, 2013 in Korea, the entire content of which is incorporated herein by reference. In addition, this non-provisional application claims priority in countries, other than the U.S., with the same reason based on the Korean patent application, the entire content of which is hereby incorporated by reference.

1. A photoacoustic imaging probe, comprising:
a light transmitting unit configured to transmit light to a target subject to be inspected; and
an acoustic-wave receiving unit configured
to receive an acoustic wave generated from the target subject by being irradiated with the light, and
to accommodate the light transmitting unit therein in a manner that a first side of the light transmitting unit is exposed to outside.

2. The photoacoustic imaging probe according to claim 1, wherein the light transmitting unit includes a light guide unit and at least one light unit.

3. The photoacoustic imaging probe according to claim 2, wherein the at least one light unit is configured to operate based on an electrical signal.

4. The photoacoustic imaging probe according to claim 2, wherein the at least one light unit is disposed in proximity of an opposing side of the first side.

5. The photoacoustic imaging probe according to claim 2, wherein the light guide unit includes an optically transparent material.

6. The photoacoustic imaging probe according to claim 2, wherein the light guide unit is tapered from the opposing side toward the first side.

7. The photoacoustic imaging probe according to claim 1, wherein
the acoustic-wave receiving unit includes a lens layer, and
the first side is disposed at a centerline of a surface of the lens layer.

8. The photoacoustic imaging probe according to claim 1, wherein
the acoustic-wave receiving unit includes a plurality of oscillators arranged at intervals along a first direction, and
the first side is formed in an elongated manner along the first direction.

9. A medical apparatus employing an acoustic wave, the medical apparatus comprising the photoacoustic imaging probe according to claim 1.

10. A method of manufacturing a photoacoustic imaging probe, the method comprising:
laminating a piezoelectric member and an acoustic-impedance matching layer on a sound absorbing layer, the sound absorbing layer, the piezoelectric member, and the acoustic-impedance matching layer laminated in this order defining a laminated member;
multi-dicing the laminated member in a second direction perpendicular to a first direction along which a plurality of oscillators is arranged at intervals;
forming a groove elongated along the first direction on a diced laminated member;
inserting a light guide unit into the groove; and
forming a lens layer so that a first side of the light guide unit is exposed to outside.

11. The method according to claim 10, wherein the inserting includes inserting the light guide unit in a manner that the light guide unit protrudes from a surface of the acoustic-impedance matching layer.

12. The method according to claim 10, further comprising forming at least one through-hole on a bottom of the groove after forming the groove.

13. A medical apparatus employing an acoustic wave, the medical apparatus comprising a photoacoustic imaging probe manufactured by the method according to claim 10.
